(19) United States
(12) Patent Application Publication
(10) Pub. No.: US 2022/0233069 A1
(43) Pub. Date: Jul. 28, 2022

(54) APPARATUS FOR ASSISTING EYELID EXAMINATION

(71) Applicant: THE CATHOLIC UNIVERSITY OF KOREA INDUSTRY-ACADEMIC COOPERATION FOUNDATION, Seoul (KR)

(72) Inventors: Dong Jin CHANG, Seoul (KR); Kyung Sun NA, Seoul (KR)

(21) Appl. No.: 17/610,281

(22) PCT Filed: Jun. 9, 2020
(86) PCT No.: PCT/KR2020/007436

§ 371 Date: Nov. 10, 2021

(30) Foreign Application Priority Data
Sep. 4, 2019 (KR) 10-2019-0109267

(51) Int. Cl. A61B 3/00; A61B 3/10

(52) U.S. Cl. CPC A61B 3/0083; A61B 3/10

(57) ABSTRACT

An apparatus for assisting an eyelid examination according to an embodiment of the present disclosure assists eversion of an eyelid in order to observe conjunctiva or meibomian glands of the eyelid. The apparatus may include a clamping part configured to hold eyelid or eyelashes around the eyelid, a pressing part having one side connected to the clamping part and the other side in contact with an upper boundary portion of an upper eyelid plate formed along an eyeball line of the eyelid, and a handle part having one side connected to the clamping part and the other side extending while having an angle with the clamping part so that a force for the eyelid eversion is applied to the clamping part at a location spaced apart from the eyelid.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2020/007436, filed Jun. 9, 2020, which claims priority to the benefit of Korean Patent Application No. 10-2019-0109267 filed in the Korean Intellectual Property Office on Sep. 4, 2019, the entire contents of which are incorporated herein by reference.

### BACKGROUND

### 1. Technical Field

The present disclosure relates to an apparatus for assisting an eyelid examination, and more particularly, to an apparatus for assisting an eyelid examination with an improved structure enabling easy eversion with one hand to improve user convenience when an upper eyelid is everted to observe the conjunctiva or meibomian glands of the upper eyelid.

### 2. Background Art

In general, an eyelid examination is performed by observation of the conjunctiva and meibomian glands of an upper eyelid. This observation requires a process of everting an eyelid.

In other words, as well shown in FIG. 1, in general eyelid eversion, an upper boundary portion A of an upper eyelid plate of an eyelid is pressed with a middle finger while gently pulling the eyelashes or eyelid with a thumb and an index finger (processes (a) and (b)) or is pressed using a cotton swab when the middle finger is difficult to use (process (c)), and then the eyelid is turned inside out while applying appropriate pressure (process (d)).

In such a conventional eyelid eversion method, accurate eversion is not achieved frequently because several fingers should be used or a cotton swab should be used with the other hand, and the help of another assistant is needed for observation of the conjunctiva or meibomian glands.

Of course, a technique for holding eyelashes using a tool without holding eyelashes with fingers has been developed and used. However, aside from the tool, a separate tool is still required to press the upper boundary portion A of the upper eyelid plate, thus still causing user inconvenience.

### SUMMARY

Provided is an apparatus for assisting an eyelid examination, by which an examiner alone easily and conveniently examines an eyelid without the help of a medical assistant.

According to an aspect of the present disclosure, an apparatus for assisting an eyelid examination, which assists eversion of an eyelid in order to observe conjunctiva or meibomian glands of the eyelid, includes a clamping part configured to hold eyelid or eyelashes around the eyelid, a pressing part having one side connected to the clamping part and the other side in contact with an upper boundary portion of an upper eyelid plate formed along an eyeball line of the eyelid, and a handle part having one side connected to the clamping part and the other side extending while having an angle with the clamping part so that a force for the eyelid eversion is applied to the clamping part at a location spaced apart from the eyelid.

The pressing part may be movably provided on the clamping part, the clamping part may include a slit enabling a movement of the pressing part, and the apparatus may further include an elastic member configured to elastically press the pressing part toward the upper boundary portion of the upper eyelid plate when the pressing part is in contact with the upper boundary portion of the upper eyelid plate.

The pressing part may include a pin part inserted into the slit and elastically pressed by the elastic member, and a pressing part extending from the pin part and configured to apply a pressing force delivered to the pin part to the upper boundary portion of the upper eyelid plate.

The clamping part and the handle part may be arranged to form an angle with each other.

The clamping part may include a pair of clamping members hinged to each other.

An apparatus for assisting an eyelid examination, according to an embodiment of the present disclosure, configured as above, may evert an eyelid by securing a pivot point on an upper boundary portion of an upper eyelid plate of the eyelid by using a pressing part connected to a clamping part while holding the eyelashes or eyelid by using the clamping part, and then holding a handle part extending from the clamping part with a thumb and an index finger and rotating the handle part. Therefore, an examiner alone may easily and conveniently observe the conjunctiva and meibomian glands of the eyelid.

### BRIEF DESCRIPTION OF THE DRAWINGS

FIG. 1 is a view for explaining a general eyelid examining method.

FIG. 2 is a perspective view of an apparatus for assisting an eyelid examination, according to an embodiment of the present disclosure.

FIG. 3 is a cross-sectional view of an embodiment of the present disclosure.

FIGS. 4 through 6 are views for explaining an eyelid eversion process according to an embodiment of the present disclosure.

FIG. 7 is a perspective view of an apparatus for assisting an eyelid examination, according to another embodiment of the present disclosure.

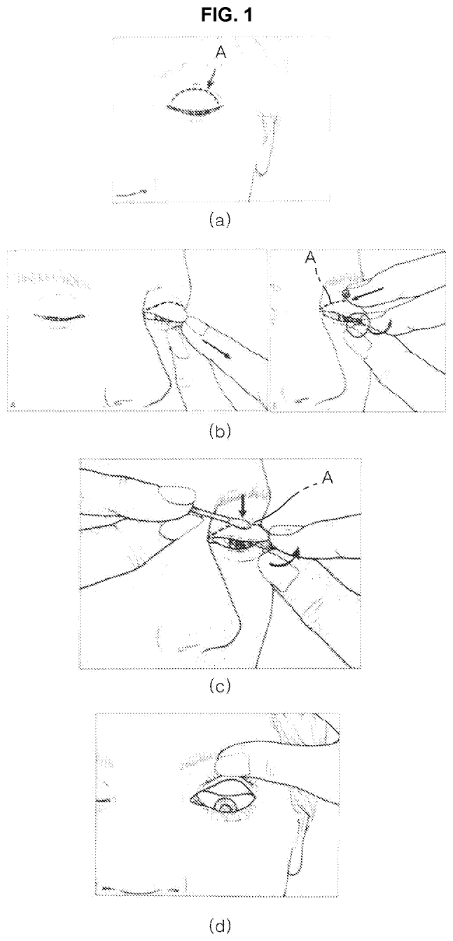

### DETAILED DESCRIPTION

In the following description, in order to clarify the understanding of the present invention, descriptions of well-known technologies for the features of the present invention will be omitted. The following embodiment are detailed descriptions to help the understanding of the present invention, and it will be understood that the scope of the present invention is not limited. Accordingly, an equivalent invention that performs the same function as the present invention will also fall within the scope of the present invention.

In addition, in the following description, the same reference numerals or symbols means the same configuration, and unnecessary redundant description and description of known technology will be omitted. Moreover, a description of each embodiment of the present invention that overlaps with the description of the technology that is the background of the present invention will also be omitted.

An apparatus for assisting an eyelid examination, according to an embodiment of the present disclosure, will now be described in detail with reference to the accompanying drawings.

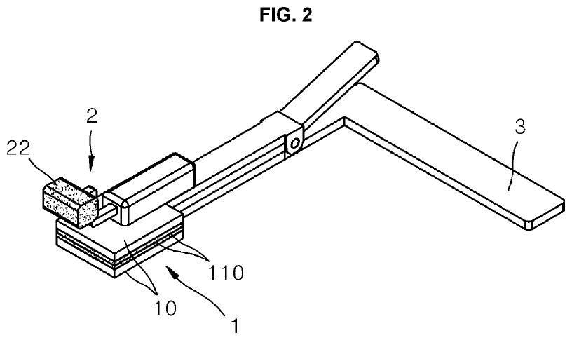

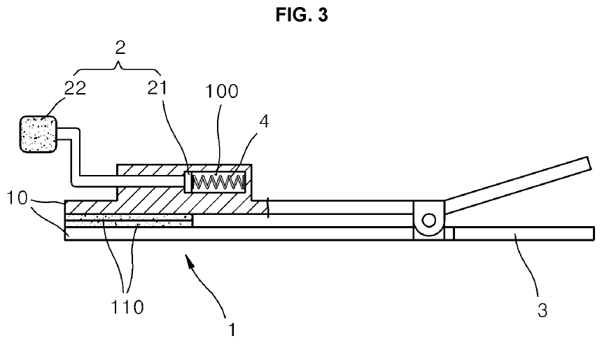

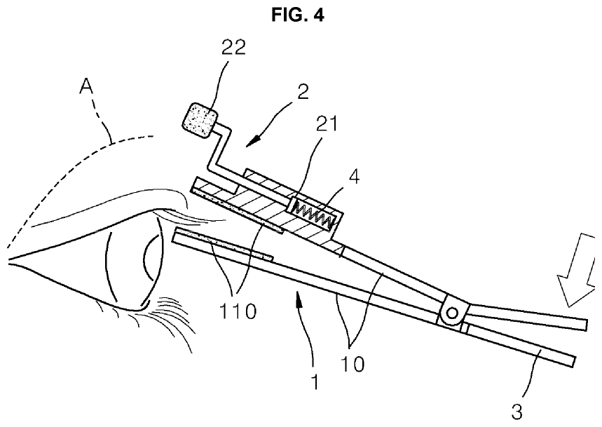

FIG. 2 is a perspective view of an apparatus for assisting an eyelid examination, according to an embodiment of the present disclosure. FIG. 3 is a cross-sectional view of an embodiment of the present disclosure. FIGS. 4 through 6 are views for explaining an eyelid eversion process according to an embodiment of the present disclosure.

The apparatus for assisting an eyelid examination, according to an embodiment of the present disclosure, assists eversion of an eyelid in order to observe the conjunctiva or meibomian glands of the eyelid, and thus includes a clamping part 1, a pressing part 2, and a handle part 3, as shown in FIGS. 2 and 3.

The clamping part 1, which holds an eyelid or the eyelashes around the eyelid as shown in FIG. 4, may be configured to have a structure enabling eyelash clamping. However, the clamping part 1 preferably includes a contact pad 110 made of a silicone material harmless to the human body so that clamping may be performed smoothly.

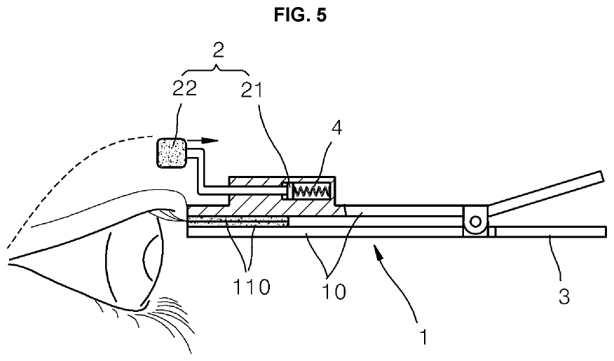

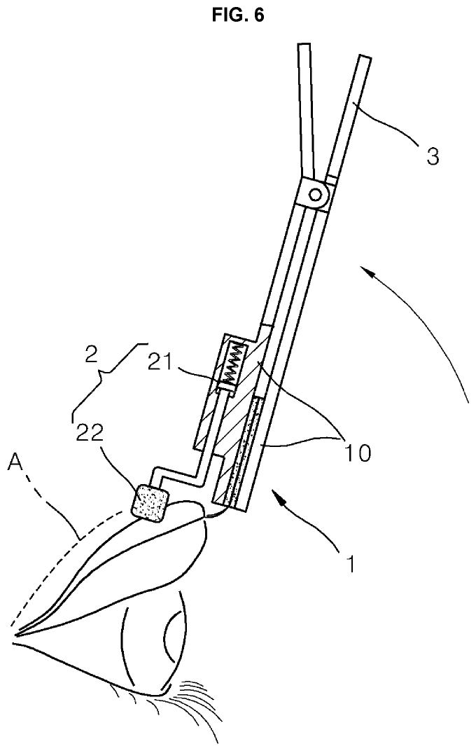

As shown in FIGS. 5 and 6, the pressing part 2 has one side connected to the clamping part 1 and the other side being selectively in contact with an upper boundary portion A of an upper eyelid plate formed along an eyeball line of the eyelid. The pressing part 2 enables formation of a pivot point for eversion according to the present embodiment in the eyelid, thereby achieving smooth eyelid eversion based on the present embodiment.

The handle part 3 has one side connected to the clamping part 1 and the other side extending while having an angle with the clamping part 1, as well shown in FIG. 2, and thus applies a force for the eyelid eversion to the clamping part 1 at a location spaced apart from the eyelid.

The apparatus for assisting an eyelid examination, according to an embodiment of the present disclosure, having this structure, may evert the eyelid by securing a pivot point on the upper boundary portion A of the upper eyelid plate of the eyelid by using the pressing part 2 connected to the clamping part 1 while holding the eyelashes or eyelid by using the clamping part 1, and then holding the handle part 3 extending from the clamping part 1 with a thumb and an index finger and rotating the handle part 3. Therefore, an examiner alone may easily and conveniently observe the conjunctiva and meibomian glands of the eyelid.

The pressing part 2 may be configured such that a relative position with respect to the clamping part 1 is fixed. However, as shown in FIG. 6, when the present embodiment is rotated using the pressing part 2 as a pivot point, the pressing part 2 may be movably provided on the clamping part 1 in order to prevent excessively strong pressure from being applied to the eyelid.

In other words, when the pressing part 2 and the clamping part 1 rotate from a state as in FIG. 5 to a state as in FIG. 6, an interval between respective leading ends of the pressing part 2 and the clamping part 1 due to folding of the eyelid decreases, and thus, when movement of the pressing part 2 is not free by the variation in the interval, strong pressure may be applied to the eyelid. Thus, the present embodiment may address this problem by enabling the pressing part 2 to move in a direction indicated by the arrow of FIG. 5.

The clamping part 1 preferably includes a slit 100 enabling movement of the pressing part 2 with respect to the clamping part 1.

Because a pivot point may be formed only when the pressing part 2 presses the upper boundary portion A of the upper eyelid plate of the eyelid with appropriate pressure, the present embodiment preferably further includes an elastic member 4 elastically pressing the pressing part 2 toward the upper boundary portion A of the upper eyelid plate when the pressing part 2 is in contact with the upper boundary portion A of the upper eyelid plate.

The present embodiment having this structure enables not only pivot point formation according to the present embodiment by applying an elastic force to the pressing part 2 but also enables movement of the pressing part 2 with respect to the clamping part 1, thereby preventing adverse effects on eye health due to application of strong pressure resistance to the upper boundary portion A of the upper eyelid plate during rotation of the pressing part 2.

The pressing part 2 exhibiting this function may be implemented in various structures and shapes. However, for simplification of a structure, the pressing part 2 preferably includes a pin part 21 inserted into the slit 100 and elastically pressed by the elastic member 4, and a pressing part 22 extending from the pin part 21 and applying a pressing force delivered to the pin part 21 to the upper boundary portion A of the upper eyelid plate.

In order for a user to easily operate the present embodiment with one hand, the clamping part 1 and the handle part 3 are preferably arranged to form an angle with each other

In other words, when the clamping part 1 holding the eyelashes or eyelid forms an angle with the handle part 3 applying an eversion force according to the present embodiment, a rotation center of the handle part 3 and a rotation center of the clamping part 1 may be formed to be approximately parallel to each other, and thus the eyelid may be smoothly everted even at a remote location spaced apart from the eyelid.

The clamping part 1 includes a pair of clamping members 10 hinged to each other, and the present embodiment preferably includes a coil spring disposed on a hinge shaft portion such that respective leading ends of the clamping members 10 may come into contact with each other by an elastic force.

The present embodiment having this structure is configured to release clamping of the eyelashes or eyelid by the clamping part 1 by applying pressure in a direction indicated by the arrow of FIG. 4 by using a finger and achieve clamping of the eyelashes or eyelid by the clamping part 1 by releasing the pressure in the direction indicated by the arrow of FIG. 4, thereby further improving user convenience.

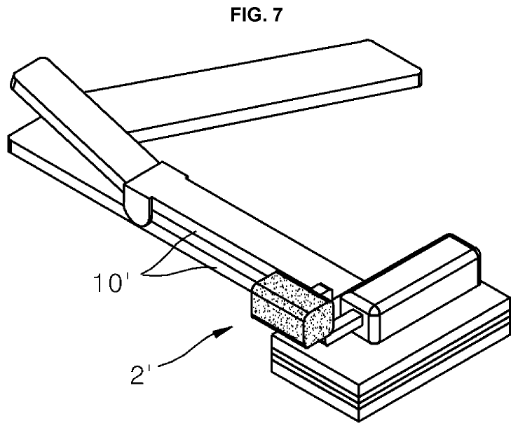

FIG. 7 is a perspective view of an apparatus for assisting an eyelid examination, according to another embodiment of the present disclosure.

In contrast with the previous embodiment in which the clamping members 10 and the pressing part 2 are disposed on the same axis, the embodiment shown in FIG. 7 has a structure in which clamping members and a pressing part are disposed orthogonally to each other.

The present embodiment having such a structure may stably perform an eversion operation of eyelashes or an eyelid.

Although various embodiments of the present disclosure have been described above, the present embodiments and the drawings attached to the present specification merely show a part of the technical spirit included in the present disclosure. It will be apparent that modifications and specific embodiments that can be easily inferred by those skilled in the art within the scope of the technical idea are included in the scope of the present disclosure.

1. An apparatus for assisting an eyelid examination, which assists eversion of an eyelid in order to observe conjunctiva or meibomian glands of the eyelid, the apparatus comprising:
a clamping part configured to hold eyelid or eyelashes around the eyelid;
a pressing part having one side connected to the clamping part and the other side in contact with an upper boundary portion of an upper eyelid plate formed along an eyeball line of the eyelid; and
a handle part having one side connected to the clamping part and the other side extending while having an angle with the clamping part so that a force for the eyelid eversion is applied to the clamping part at a location spaced apart from the eyelid.

2. The apparatus of claim 1, wherein the pressing part is movably provided on the clamping part,
the clamping part includes a slit enabling a movement of the pressing part, and
the apparatus further comprises an elastic member configured to elastically press the pressing part toward the upper boundary portion of the upper eyelid plate when the pressing part is in contact with the upper boundary portion of the upper eyelid plate.

3. The apparatus of claim 2, wherein the pressing part includes a pin part inserted into the slit and elastically pressed by the elastic member, and a pressing part extending from the pin part and configured to apply a pressing force delivered to the pin part to the upper boundary portion of the upper eyelid plate.

4. The apparatus of claim 1, wherein the clamping part and the handle part are arranged to form an angle with each other.

5. The apparatus of claim 1, wherein the clamping part includes a pair of clamping members hinged to each other.
